(19) United States
(12) Patent Application Publication
(10) Pub. No.: US 2024/0307090 A1
(43) Pub. Date: Sep. 19, 2024

(54) DETACHABLE MECHANISM FOR VERIFYING DEVICE ACCESS

(71) Applicant: BOSTON SCIENTIFIC SCIMED, INC., Maple Grove, MN (US)

(72) Inventors: Cristina ROMANY, Orlando, FL (US); Robb Morse GAVALIS, Westborough, MA (US); Alexandra HAUGEN, Westborough, MA (US)

(21) Appl. No.: 18/422,653

(22) Filed: Jan. 25, 2024

Related U.S. Application Data
(60) Provisional application No. 63/490,329, filed on Mar. 15, 2023.

(51) Int. Cl. A61B 17/34; A61B 17/00; A61B 90/00; A61B 90/57

(52) U.S. Cl. CPC A61B 17/3421; A61B 90/37; A61B 90/57; A61B 2017/00022; A61B 2017/00818; A61B 2017/3445; A61B 2090/376

(57) ABSTRACT

A luer device includes Connector with a body and a lumen and Clip with exterior and interior portions. The body includes a distal portion to be coupled to cannula and a proximal portion to slidably receive a medical device. The exterior portion extends along longitudinal Axis from proximal to distal ends and includes a channel. A longitudinal slot extends along the exterior portion so that the channel is open to and in communication with a clip exterior via the slot. The interior portion extends along an interior surface of the channel defining jaws and formed of a material which, when Clip is moved from open toward closed configurations, in an operative position, is pressed against the device and Connector to seal a proximal end of Connector and direct fluid distally therethrough and through a space extending between an interior surface of cannula and an exterior surface of the device.

### PRIORITY CLAIM

The present disclosure claims priority to U.S. Provisional Patent Application Ser. No. 63/490,329 filed Mar. 15, 2023; the disclosure of which is incorporated herewith by reference.

### BACKGROUND

Endoscopic Ultrasound (EUS) is a minimally invasive procedure performed with a specialized endoscope that uses high frequency soundwaves to visualize internal organs (e.g., the digestive (gastrointestinal) tract and nearby structures). According to one application, EUS may be used to facilitate biliary drainage after the failure, for example, of a traditional endoscopic retrograde cholangiopancreatology (ERCP), which utilizes contrast dye and X-rays to identify and treat blockages within the ducts. EUS may be used to gain guidewire access into the common bile or pancreatic ducts. Successful EUS access procedures depend on the initial duct puncture and access cannula stability within the duct while passing a guidewire.

According to some current procedures, once access has been gained into the duct or other anatomy via a sharp or other needle advanced from the access cannula to pierce the duct or tissue wall, the sharp is removed so that a contrast or saline solution may be injected through the access cannula to the target site to enable the verification of a position of the access cannula within the duct via fluoroscopic visualization. After the desired position of the access cannula has been verified, a guidewire is passed therethrough to a target site to facilitate the advancement to the target site of other treatment/diagnosis devices. For cases in which it is determined via fluoroscopic visualization that the access cannula has not penetrated into the duct as desired, the sharp must be reinserted through the access cannula to repeat the process of positioning the cannula resulting in loss of time and creating the potential for losing the position of the access cannula relative to the duct.

### SUMMARY

The present disclosure relates to a luer device for accessing a biliary duct. The device includes a connector and a clip. The connector includes a body extending longitudinally from a proximal end to a distal end. The connector includes a lumen extending therethrough. The body of the connector includes a distal portion sized, shaped, and configured to be coupled to a proximal end of an access cannula and a proximal portion of the body sized, shaped, and configured to slidably receive a medical device received within the access cannula.

The clip is configured to be clipped over the proximal portion of the connector and a portion of the medical device extending therefrom. The clip includes an exterior portion and an interior portion. The exterior portion extends along a longitudinal axis from a proximal end to a distal end and includes a channel extending therethrough along the longitudinal axis. A longitudinal slot extends along an entire length of the exterior portion so that the channel is open to and in communication with an exterior of the clip via the longitudinal slot. The interior portion extends along an interior surface of the channel defining jaws and formed of a material which, when the clip is moved from an open configuration toward a closed configuration, in an operative position, is pressed against the medical device and the connector to seal the proximal end of the connector and direct fluid distally therethrough and through a space extending between an interior surface of the access cannula and an exterior surface of the medical device.

In an embodiment, the longitudinal slot defines a pair of jaws movable between the open configuration, in which longitudinal edges defining the jaws are moved away from one another, and the closed configuration, in which the longitudinal edges are moved toward one another to draw longitudinal edges of the interior portion toward one another to seal the proximal end of the connector.

In an embodiment, the clip is biased toward the closed configuration.

In an embodiment, the exterior portion of the clip includes a pair of wings, each of the wings extending laterally from a corresponding one of the jaws, in a direction away from the longitudinal edges of the jaws so that, when the pair of wings are drawn toward one another the jaws are moved away from one another toward the open configuration, and when the pair of wings are released, the jaws are permitted to revert toward the closed configuration.

In an embodiment, the interior portion is formed of a softer material then the exterior portion of the clip.

In an embodiment, the connector includes a fluid connection port in fluid connection with the lumen of the connector via a connecting tube so that fluid injected via the fluid connection port.

In an embodiment, the proximal portion of the connector has a smaller cross-sectional area than the distal portion of the connector.

In an embodiment, the distal portion of the connector includes a threading along an interior surface thereof for threadedly engaging a corresponding threading along the proximal end of the access cannula.

In an embodiment, the exterior portion of the clip is rotatable relative to the interior portion to move the clip from the open configuration toward the closed configuration.

In addition, the present disclosure relates to a luer device which includes a proximal component, a distal component, an interior portion and a tubular member. The proximal component extends along a longitudinal axis from a proximal end to a distal end and includes a channel extending therethrough. A longitudinal slot extends along an entire length of the proximal component so that the channel thereof is open to and in communication with an exterior of the proximal component via the longitudinal slot. The longitudinal slot defines a pair of jaws movable between an open configuration, in which longitudinal edges defining the jaws are moved away from one another, and a closed configuration, in which the longitudinal edges are moved toward one another.

The distal component is rotatably longitudinally aligned with and rotatably coupled to the proximal component so that the distal component is rotatable relative thereto about the longitudinal axis thereof, the distal component extending from a proximal end to a distal end and including a channel extending therethrough. A longitudinal slot extends along an entire length of the distal component so that the channel thereof is open to and in communication with an exterior of the distal component via the longitudinal slot thereof, a distal portion of the distal component including a threading along an interior thereof for threadedly engaging the proximal end of an access cannula.

The interior portion extends along an interior surface of the channel of the proximal component defining the jaws and along an interior surface along an unthreaded proximal portion of the distal component, the interior portion formed of a material which, when pressed against a medical device, forms a seal thereabout.

The tubular member is received within the interior portion, along a distal portion thereof, a distal end of the tubular member corresponding to a distal end of the interior portion so that, when the proximal portion is in the closed configuration in an operative configuration over the medical device, and the distal component is coupled to the access cannula and the medical device is received within the tubular member, a proximal end of the tubular member is sealed and fluid is directed distally through an access channel, about the medical device.

In an embodiment, the interior portion extends along the interior surface of the proximal component so that, when the proximal portion is in the open configuration longitudinal edges of the interior portion are separated from one another and, when the proximal portion is in the closed configuration, longitudinal edges of the interior portion are drawn toward one another.

In an embodiment, the proximal component includes a pair of wings, each of the wings extending laterally from a corresponding one of the jaws, in a direction away from the longitudinal edges of the jaws so that, when the pair of wings are drawn toward one another the jaws are moved away from one another toward the open configuration, and when the pair of wings are released, the jaws are permitted to revert toward the closed configuration.

In an embodiment, the interior portion is formed of a softer material than an exterior portion of a clip.

In an embodiment, the device further includes a fluid connection port in fluid connection with the tubular member, the fluid connection port configured to extend exterior to the proximal component, when the proximal component is in the closed configuration.

In an embodiment, the distal component is rotatable relative to the interior portion so that, as the distal component is moved distally relative to the access cannula during a threaded engagement therebetween, the distal end of the interior portion is pressed distally against the proximal end of the access cannula to direct fluid flow distally through the tubular member and distally through a space extending between an interior of the access channel and an exterior surface of the medical device.

Furthermore, the present disclosure relates to a method for treating a biliary duct. The method includes coupling a connector to a proximal end of an access cannula, which includes a medical device housed therein and a distal end which has been inserted into a target duct, such that a distal portion of the connector is engaged with the proximal end of the access cannula and a portion of the medical device extending proximally from the access cannula is slidably received within a proximal portion of the connector; clipping a clip over a proximal portion of the connector and a portion of the device extending proximally therefrom, the clip including an interior portion which, when the clip is in a clipped configuration, is pressed against the medical device and the connector to seal the proximal end of the connector; and injecting a fluid, via a fluid injection port that is in fluid communication with the connector, into a channel of the connector, the fluid being directed distally through the connector, since the proximal end of the connector is sealed, and distally through a space extending between an interior surface of an access channel and an exterior surface of the medical device.

In an embodiment, the method further includes confirming, via fluoroscopic visualization, whether the distal end of the access cannula is positioned within the target duct, as desired.

In an embodiment, clipping the clip includes moving the clip from an open configuration toward a closed configuration, the clip including an exterior portion extending along a longitudinal axis from a proximal end to a distal end and including a channel extending therethrough along the longitudinal axis, a longitudinal slot extending along an entire length of the exterior portion so that the channel is open to and in communication with an exterior of the clip via the longitudinal slot, the interior portion extending along an interior surface of the channel defining jaws and formed of a material which, when the clip is moved from an open configuration toward a closed, clipping configuration, longitudinal edges of the interior portion are drawn toward one another and the interior portion is pressed against the medical device and the connector received therein to form a seal thereabout.

In an embodiment, moving the clip between the open and closed configurations includes drawing a pair of wings, each of which extend laterally from a corresponding one of the jaws, toward one another to move the jaws of the clip away from one another toward the open configuration, and releasing the wings so that the jaws are permitted to revert toward the closed configuration.

In an embodiment, the medical device is one of a needle and a guidewire.

### BRIEF DESCRIPTION OF THE DRAWINGS

FIG. 1 shows a perspective view of a system according to an exemplary embodiment of the present disclosure;

FIG. 2 shows a cross-sectional view of the system according to FIG. 1;

FIG. 3 shows a side view of a proximal portion of an access cannula and medical device received therein, according to the system of FIG. 1;

FIG. 4 shows a side view of a distal portion of the access cannula and medical device inserted into a target duct and/or anatomy, wherein the medical device is a needle and fluid is injected through the access cannula about the needle, according to an exemplary embodiment of the system of FIG. 1;

FIG. 5 shows a side view of a distal portion of an access cannula and medical device, wherein the medical device is a guidewire and fluid is injected through the access cannula about the guidewire, according to another exemplary embodiment of the system of FIG. 1;

FIG. 6 shows a partially transparent side view of a connector of a luer device, according the system of FIG. 1;

FIG. 7 shows a plan view of a clip of the luer device, clipped over the medical device, according to the system of FIG. 1;

FIG. 8 shows a plan view of the clip of the luer device, in an open configuration, according to the system of FIG. 1;

FIG. 9 shows a plan view of the clip of the luer device, in a closed configuration, according to the system of FIG. 1;

FIG. 10 shows a plan view of a clip of a luer device according to another exemplary embodiment of the present disclosure, in an open configuration;

FIG. 11A shows a plan view of the clip of the luer device according to the system of FIG. 10, in a closed configuration according to a first embodiment;

FIG. 11B shows a plan view of the clip of the luer device according to the system of FIG. 10, in a closed configuration according to a second embodiment;

FIG. 12 shows a perspective view of a system according to another exemplary embodiment of the present disclosure;

FIG. 13 shows a cross-sectional view of the system according to FIG. 12;

FIG. 14 shows a cross-sectional view of a luer device according to another exemplary embodiment of the present disclosure attached to a medical device and an access cannula of the system of FIG. 12; and

FIG. 15 shows a perspective view of the luer device of FIG. 14.

### DETAILED DESCRIPTION

The present disclosure may be further understood with reference to the following description and appended drawings, wherein like elements are referred to with the same reference numerals. The present disclosure relates to endoscopic ultrasound (EUS) systems and procedures, and, in particular, relates to a device (e.g., a luer device) connectable to an access cannula to facilitate fluid insertion through the access cannula, about a medical device received therein. Exemplary embodiments describe the device configured to be coupled to a proximal end of an access cannula and the medical device received therein so that a fluid solution (e.g., contrast or saline solution or echogenic media) may be injected distally through a space between an interior surface of the access cannula and an exterior surface of the medical device to facilitate confirmation of proper placement of the access cannula via fluoroscopic or EUS visualization.

Although some of the exemplary embodiments specifically describe the luer device as being configured for use with an access cannula for accessing a bile duct, it will be understood by those of skill in the art that the luer device of the present disclosure may also be used with a variety of other endoscopic devices (e.g., catheter) in which it is desired to inject a fluid therethrough, about a medical device received therein, to a target area within a body. Further, although the exemplary embodiments describe a luer device connectable to an access cannula, it will be understood by those of skill in the art that the present disclosure also relates to a luer accessory device intended to interact with a luer coupled to an access cannula to facilitate fluid insertion through the access cannula. For example, in a further exemplary embodiment, the luer accessory device may squeeze fit around and interface with a luer connected to an access cannula. It should also be noted that the terms “proximal” and “distal,” as used herein, are intended to refer to a direction toward (proximal) and away from (distal) a user of the device (e.g., physician).

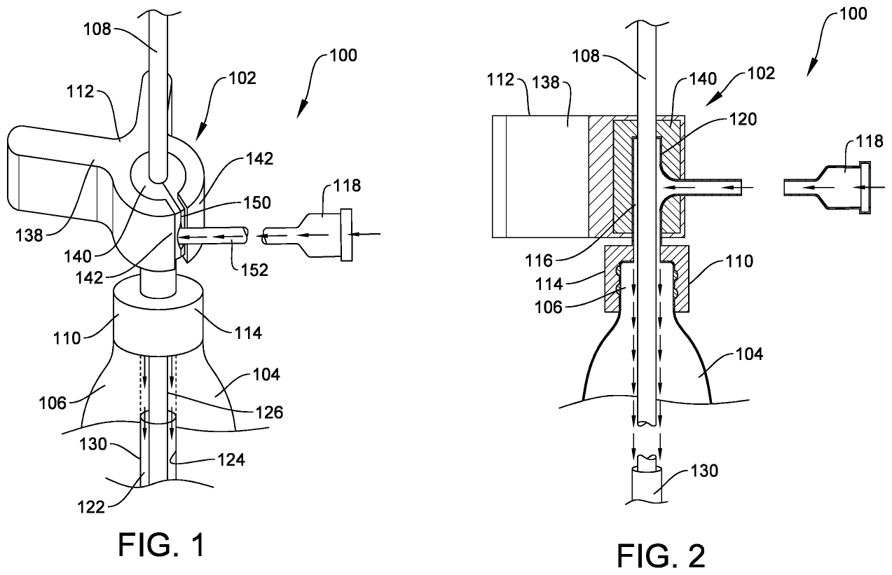

As shown in FIGS. 1-9, a system 100 according to an exemplary embodiment of the present disclosure comprises a device 102 (e.g., a luer device) configured to be coupled to a proximal end 106 of an access cannula 104 and a medical device 108 (e.g., sharp, needle, guidewire) received therein to facilitate injection of a fluid 14 through the access cannula 104, about the medical device 108. Injection of the fluid 14 (e.g., contrast agent, saline, echogenic media) allows a user (e.g., physician) to determine via fluoroscopic or EUS visualization whether a distal end 107 of the access cannula 104 has been positioned as desired within a target duct 10 or other anatomic location. According to an exemplary embodiment, as shown in FIGS. 1 and 2, the device 102 further comprises a connector 110 including a fluid injection port 118 and a clip 112.

The connector 110 includes a distal portion 114 configured to be coupled to a proximal end 106 of the access cannula 104 and a proximal portion 116 sized, shaped and configured to slidably receive a portion of the medical device 108 extending proximally from the access cannula 104 therein. In an exemplary embodiment, the fluid injection port 118 extends laterally from the proximal portion 116. The clip 112 is configured to be mounted over the proximal portion 116 of the connector 110 (e.g., over a proximal end 120 thereof and a portion of the medical device 108 extending proximally out of the connector 110) to form a seal so that fluid 14 may be injected through the fluid injection port 118 to pass distally through an annular space 122 between an interior surface 124 of the access cannula 104 and an exterior surface 126 of the medical device 108. If, under fluoroscopic visualization, the fluid 14 is seen to be filling the target duct 10 or another target anatomical structure, successful placement of the access cannula 104 is confirmed. If, however, visualization shows that the fluid 14 is not filling the target duct or anatomy, it is an indication that the access cannula 104 has not been positioned within the target duct 10 or anatomy, as desired.

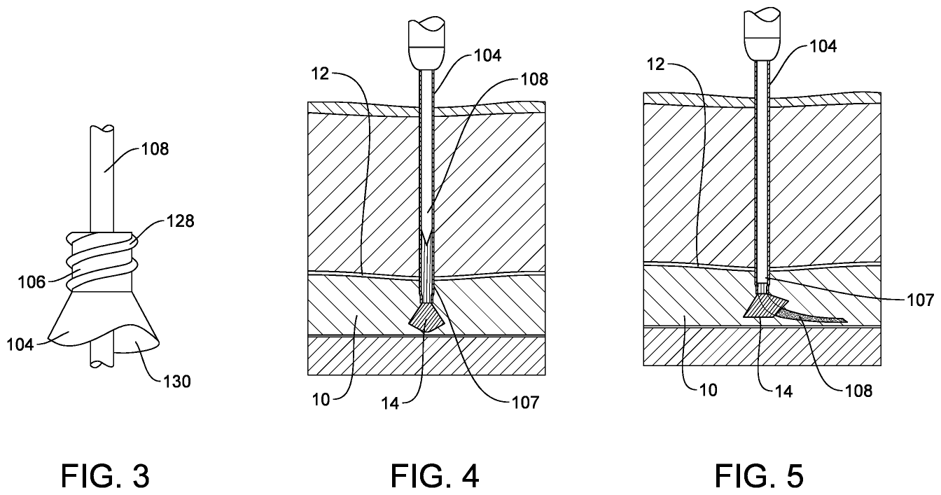

The device 102 is configured to be connected to any standard access cannula 104 configured for accessing a target duct 10 or other anatomical structure (e.g., in an EUS procedure such as biliary drainage). The access cannula 104 extends longitudinally from the proximal end 106 to a distal end 107 and includes a channel 130 extending therethrough. As shown in FIG. 3, the proximal end 106 includes an engaging element such as, for example, threading 128 extending therealong. As will be described in further detail below, the threading 128 is configured to engage corresponding threading on the distal portion 114 of the connector 110. The channel 130 is configured to receive a medical device 108 such as, for example, a sharp or needle, which pierces a wall 12 of the target duct 10 so that the distal end of the access cannula 104 may be inserted therethrough into the target duct or into another target anatomical structure. Upon proper placement of the access cannula 104 in the target duct 10, the sharp or needle may be removed so that another medical device (e.g., a guidewire) may be inserted through the channel 130 and into the target duct 10 to provide a guide along which, for example, a catheter may be inserted to provide drainage of the target duct 10.

As described above, upon placement of the access cannula 104, it may be desired to confirm that the distal end of the access cannula 104 has been successfully positioned within the target duct 10 via fluoroscopic visualization. Current procedures require the user to remove the sharp or needle prior to injection of the fluid solution 14 for fluoroscopic visualization. If, however, the fluoroscopic visualization indicates that the access cannula has not been properly positioned within the target duct 10, the user will be required to reinsert the sharp or needle to re-pierce the tissue of the duct 10 so that the access cannula may be inserted properly therein. This re-insertion of the sharp loses valuable time and risks losing the position of the access cannula 104 adjacent to the target duct 10 as the users manipulate the device.

As shown in FIG. 4, the device 102 permits injection of the fluid solution 14 for fluoroscopic visualization through the access cannula 104, around the sharp of the needle while the needle is received in the access cannula 104. In addition, for example when an access cannula 104 that has been inserted properly into a duct 10 is at risk of being moved out of the target duct (e.g., due to a limited depth of insertion with smaller ducts or when anatomical movement may move the access cannula 104 out of a previously accessed duct), the user (e.g., physician) may desire to re-confirm the proper placement of the cannula before proceeding with the drainage procedure. As shown in FIG. 5, the device 102 of the present disclosure permits injection of a fluid solution 14 through the channel 130 of the access cannula 104, even while the medical device 108 such as, for example, the sharp, needle, or a guidewire, is still received therein—i.e., fluid may be supplied to the distal end of the access cannula 104 to confirm its position without removing a guidewire from the access cannula 104. Those skilled in the art will understand that, once a guidewire has been successfully placed in a target duct or other target anatomical structure, it is desirable to maintain this placement so that additional devices may be inserted into the target structure without removing and repeating the placement of the guidewire.

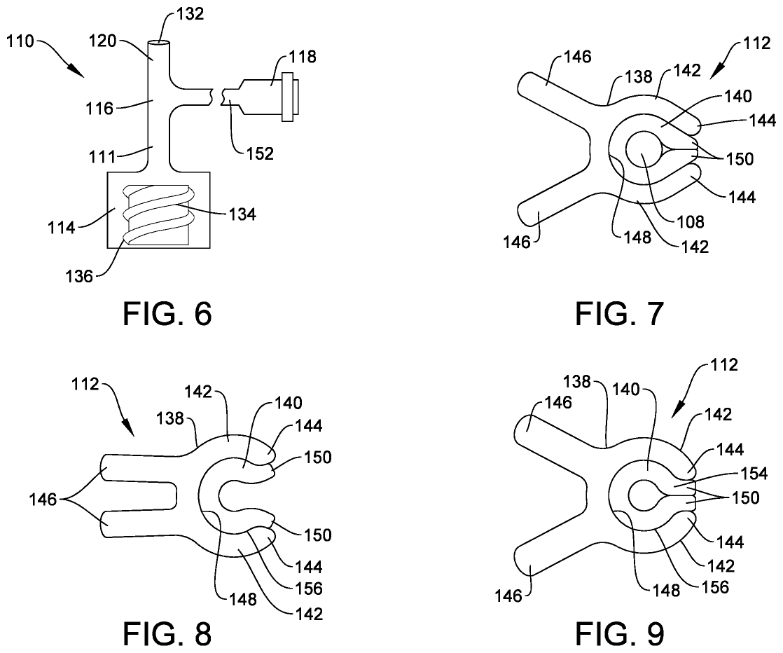

According to an exemplary embodiment, the connector 110 of the device 102 is configured to be coupled to both the access cannula 104 and the medical device 108 received in the access cannula 104. As shown in FIG. 6, the connector 110 includes a body 111 extending longitudinally from a proximal end 120 to a distal end 136 and including a lumen 132 extending therethrough. The body 111 of the connector 110 includes both the distal portion 114, which is configured to engage the proximal end 106 of the access cannula 104, and the proximal portion 116, which is configured to slidably engage the medical device 108.

The lumen 132 extends through both the distal portion 114 and the proximal portion 116. According to an exemplary embodiment, the proximal portion 116 has a smaller cross-sectional area (e.g., diameter) than the distal portion 114 so that a portion of the lumen 132 extending through the proximal portion 116 has a smaller cross-sectional area than a portion of the lumen 132 extending through the distal portion 114 and so that the proximal portion 116 sealingly engages the medical device 108 so that fluid introduced into the connector 110 via the fluid injection port 118 flows distally into the access cannula 104.

In an exemplary embodiment, the portion of the lumen 132 extending through the proximal portion 116 substantially corresponds in shape and size to the medical device 108 received therein. In an exemplary embodiment, the distal portion 114 is sized, shaped and configured to be placed over the proximal end 106 of the access cannula 104 and includes a threading 134 along an interior surface thereof—e.g., along a surface of the lumen 132—configured to threadedly engage a corresponding threading on the proximal end 106 of the access cannula 104 so that rotation of the distal portion 114 about a longitudinal axis threads the threading 128 onto the proximal end 106 of the access cannula 104 to sealingly connect the connector 110 to the access cannula 104. The connector 110 of this embodiment is configured to engage the access cannula 104 so that the lumen 132 of the connector 110 extends substantially coaxially relative to the channel 130.

The connector 110 further includes the fluid injection port 118, which is connected to the body 111 thereof. In an exemplary embodiment, the fluid injection port 118 is connected to the proximal portion 116 of the body 111 via, for example, a connecting tube 152, so that the fluid injection port 118 is in fluid communication with the lumen 132 of the connector body 111. The fluid injection port 118 is configured to be coupled to a fluid source such as, for example, a syringe.

The clip 112 is configured to be clipped over, for example, the proximal portion 116 of the connector 110 and the portion of the medical device 108 extending proximally from the proximal end 120 of the connector 110 to form a seal thereabout. The clip 112 therefore forms a seal about the proximal end 120 of the connector 110 and the medical device 108, as shown in FIG. 7, so that fluid 14 injected into the lumen 132 of the connector 110 via the fluid injection port 118 is prevented from escaping through the proximal end 120 and is directed along a fluid path, distally through the lumen 132 of the connector 110 and the channel 130 of the access cannula 104, through the annular space 122 extending around the medical device 108. In other words, as described above, the fluid 14 passes from the port 118 distally through the annular space 122 between the interior surface 124 of the access cannula 104 and an exterior surface 126 of the medical device 108 to exit the distal end of the access cannula 104. Thus, when fluid 14 is supplied to the annular space 122 and exits the distal end of the access cannula 104, a user observing the fluid (e.g., under fluoroscopic visualization) will see if the fluid 14 reaches the target duct 10 (i.e., if the distal end of the access cannula is positioned within the target duct 10).

According to an exemplary embodiment, as shown in FIGS. 7-9, the clip 112 includes an exterior portion 138 configured to move the clip 112 between an open configuration (shown in FIG. 8) and a closed configuration (shown in FIGS. 7 and 9), and an interior portion 140 configured to form a seal at the proximal end 120 of the connector 110. The exterior portion 138 in this embodiment is formed of material that permits deformation sufficient to permit the exterior portion 138 to be moved between an open and a closed configuration, as will be described in further detail below. In one example, the exterior portion is formed of a plastic material such as, for example, acrylic. The interior portion 140 is formed of a softer material than the exterior portion 138 and is configured to form a seal about an element or device against which it is pressed. In one embodiment, may be formed of a softer plastic material such as, for example, silicone.

The exterior portion 138 includes a pair of jaws 142 connected to one another and movable between the open configuration, in which longitudinal edges 144 of the jaws 142 are separated from one another to allow a portion of the connector 110 and the medical device 108 to be received therebetween, and the closed configuration, in which the edges 144 are drawn toward one another to press the interior portion 140 against the portion of the connector 110 and the medical device 108 received between the jaws 142. In other words, the jaws 142 are movable toward (closed configuration) and away from (open configuration) a longitudinal axis along which the exterior portion 138 extends.

In one exemplary embodiment, the jaws 142 are integrally formed with one another and may be formed of a single element that is bent so that the jaws 142 form a substantially tubular shape defining a channel 156 extending longitudinally therethrough and which is open to an exterior thereof via a longitudinal slot 154 extending along an entire length thereof. The longitudinal slot 154 is defined via the longitudinal edges 144. In an exemplary embodiment, a cross-section of the exterior portion 138 would show an interior surface 148 extending along the pair of jaws as having a substantially circular or C-shape. In an exemplary embodiment, the jaws 142 are biased toward the closed configuration. The jaws 142 are movable between the open and the closed configurations via a pair of wings 146, each of the wings 146 extending laterally from a corresponding one of the jaw 142, in a direction extending away from the longitudinal edges 144 of the jaws 142. Drawing the wings 146 toward one another moves the exterior portion 138 from the biased closed configuration toward the open configuration, and releasing the wings 146 permits the jaws 142 to revert to the closed configuration under their natural bias.

As described above, the interior portion 140 is formed of a material that is softer than the material of the exterior portion 138. The material of the interior portion 140 is chosen so that, when the clip 112 is in the closed configuration, this material forms a liquid-tight seal at the proximal end 120 of the connector 110. The interior portion 140 extends along the interior surface 148 of the jaws 142 so that longitudinal edges 150 of the interior portion 140 extend along the longitudinal edges 144 of the jaws 142. Thus, when the exterior portion 138 is moved between the open and closed configurations, the longitudinal edges 150 of the interior portion 140 are correspondingly separated from and drawn toward one another, respectively.

In particular, in the open configuration, the longitudinal edges 150 are separated from one another to permit the connector 110 and the medical device 108 to be moved past the longitudinal edges 150 between the jaws 142. In the closed configuration, the longitudinal edges 150 are pressed against one another so that the interior portion 140 extends about and is pressed against the proximal portion 116 of the connector 110 and the portion of the medical device 108 extending proximally therefrom to form a seal thereabout. In an exemplary embodiment, the clip 112 may be clipped or mounted over the connector 110 and the medical device 108 so that the fluid injection port 118 and/or the connecting tube 152 extend between the longitudinal edges 144, 150 of the exterior and interior portions 138, 140, respectively, of the clip 112.

Although the exemplary embodiments show and describe the clip 112 positioned over a substantial length of the proximal portion 116 of the connector 110, it will be understood by those of skill in the art that this is not required and that the clip 112 may be configured to extend over any desired length of the proximal portion 116 so long as the clip 112 extends over the proximal end 120 to form the desired liquid-tight seal. As described above, the seal formed at the proximal end 120 of the connector 110 prevents fluid 14 from flowing proximally out of the connector 110 so that fluid provided via the fluid injection port 118 moves distally through the connector 110 along the fluid path defined via the annular space 122 between the interior surface 124 of the channel 130 of the access cannula 104 and the exterior surface 126 of the medical device 108. Thus, fluid 14 injected through the access canula 104 moves through the access cannula 104 around any medical device 108 received therein to pass distally out of the access cannula 104. This permits the position of the distal end of the access cannula 104 to be observed (e.g., to visually confirm successful placement of the access cannula 104 within the target duct 10).

According to an exemplary method utilizing the system 100 for treating, for example, a biliary duct, the distal end 107 of the access cannula 104 is to be positioned within a target duct 10 of the patient. As will be understood by those of skill in the art, this may be achieved by advancing an endoscope through the GI tract to a position (e.g., within the stomach, duodenum, and/or the small intestine) adjacent to the target biliary duct. The access cannula 104, with the connector 110 coupled to the proximal end 106 thereof, is then advanced distally through a working channel of the endoscope and the sharp is extended distally out of the distal end 107 of the access cannula 104 and advanced to pierce the wall of the GI tract and then to pierce the wall 12 of the duct 10.

The access cannula 104 advances with the sharp to pass through the wall of the GI tract and is moved further distally until the distal end 107 of the access cannula 104 enters the duct 10. The sharp/needle is then retracted into the access cannula 104 (e.g., to minimize the risk of contact between the sharp and the far wall of the duct 10). The user then moves the connector 110 to the closed configuration to seal the connector 110 around the portion of the sharp extending out of the proximal end of the access cannula 104 and connects a source fluid 14 to the port 118 of the device 102. As described above, the clip 112 may be moved from the biased closed configuration toward the open configuration, by drawing the wings 146 of the exterior portion toward one another, so that the connector 110 and the medical device 108 may be received between the longitudinal edges 144, 150 of the exterior and interior portions 138, 140 of the clip 112. When a desired portion of the medical device 108 is received between the jaws 142, the wings 146 are released to permit the clip 112 to revert to the closed configuration under its natural bias. In the closed configuration, the interior portion 140, which lines the interior surface 148 of the exterior portion 138, is pressed against the connector 110 at the medical device 108 to form a seal at the proximal end 120 of the connector 110 as described above.

As described above, the clip 112 is clipped over the connector 110 such that the fluid injection port 118 and/or the connecting tube 152 extends between longitudinal edges 150 of the interior portion 140. It will be understood by those of skill in the art that the connector 110 is formed of a material having sufficient rigidity that it does not collapse when the interior portion 140 is sealed thereabout and/or against. Thus, fluid 14 may be injected via the fluid injection port 118, through the lumen 132 of the connector 110 and the channel 130 of the access cannula 104, about the medical device 108.

When the user desires to check the positioning of the access cannula 104, the user injects fluid 14 (e.g., contrast, saline, or echogenic media) through the channel 130 to pass along the sharp. The user can then observe via a medical procedure (e.g., fluoroscopy and/or EUS) whether the fluid 14 is supplied to the target duct 10. If so, this indicates correct placement of the access cannula 104. If the fluid 14 is observed not to fill the target duct 10, this indicates that the access cannula 104 has not been positioned within the target duct 10, as desired. The user may then move the connector 110 to the open configuration to permit the sharp to slide into and out of the access cannula 104. The user then advances the sharp again into the target duct 10 (as the sharp was not withdrawn from the access cannula 104 to supply the fluid 14) and again advance the access cannula 104 over the sharp until the distal end 107 of the access cannula 104 is positioned within the duct 10. The process of confirming the position of the access cannula 104 may then be repeated until the proper position of the access cannula 104 within the duct 10 is confirmed visually without ever removing the sharp from the access cannula 104.

Upon confirmation that the access cannula 104 has successfully accessed the target duct 10, the clip 112 may be removed and the sharp can then be withdrawn from the access cannula 104. The user may then pass another medical device 108 such as, for example, a guidewire, through the access cannula 104 into the target duct 10 to provide a guide along which other items (e.g., a catheter) may be slid into the target duct 10 to treat the duct 10. It will be understood that the connector 110 may remain coupled to the access cannula 104 during insertion of the guidewire. As described above, in some cases, it may be desirable to confirm that access to the duct 10 has not been lost during insertion of the guidewire before proceeding with treatment procedure. In these cases, the clip 112 may be clipped over the connector 110 and the guidewire, as described above to create a seal at the proximal end 120 of the connector 110 so that fluid 14 may once again be injected via the annular space 122 to confirm proper access of the access cannula 104 within the target duct. Upon confirmation that the access cannula 104 remains properly within the target duct 10, the user may proceed with treatment of the duct 10.

Although the exemplary embodiments show and describe the connector 110 as a component of the device 102 that is coupled to the access cannula 104 before insertion of the access cannula 104 into the body, it will be understood by those of skill in the art that, in another embodiment, the connector 110 may be coupled to the access cannula 104 after an initial insertion of the distal end of the access cannula 104 into the target duct 10. In yet another embodiment, the connector 110 may be integrally formed with the access cannula 104.

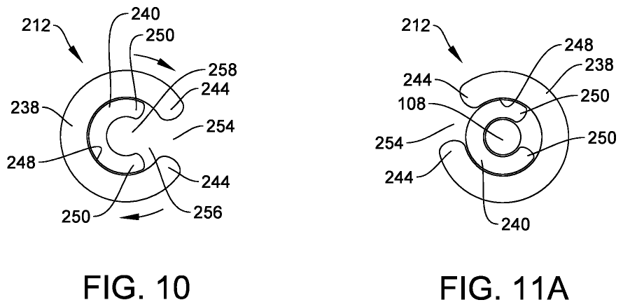

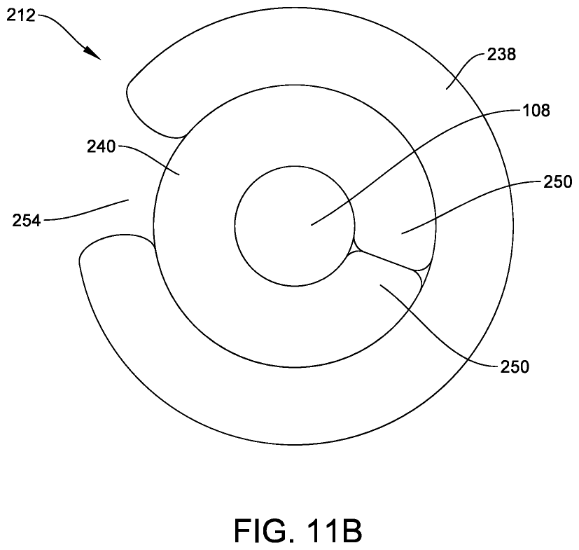

As shown in FIGS. 10-11B, a clip 212 according to another exemplary embodiment is substantially similar to the clip 112, described above, and may be used in place of the clip 112 in the device 102 according to the system 100, as described above. In particular, the clip 212 may be clipped or mounted over the proximal portion 116 and, in particular, the proximal end 120 of the connector 110, which has been coupled to a proximal end 106 of an access cannula 104, and to a portion of the medical device 108 received within the access cannula 104 and extending proximally from the connector 110, to form a seal at the proximal end 120 preventing fluid injected into the fluid injection port 118 from exiting the proximal end 120. Rather, fluid is directed distally through the annular space 122. Similarly to the clip 112, the clip 212 includes an exterior portion 238 configured to move the clip 212 between an open configuration and a closed configuration, and an interior portion 240 configured to be pressed against the connector 110 and the medical device 108 to form the liquid-tight seal thereabout.

The exterior portion 238 in this embodiment is moved between the open and closed configurations by being rotated relative to the interior portion 240 about a longitudinal axis of the exterior portion 238. In an exemplary embodiment, the exterior portion 238 is configured as an open tube including a longitudinal slot 254 extending along an entire length thereof to define longitudinal edges 244. A channel 256 defined via a shape of the exterior portion 238 is open to an exterior of the tube via the longitudinal slot 254. In one embodiment, a cross-section of the exterior portion 238 is substantially C-shaped. The exterior portion 238 may be formed of a plastic material similar to the material of which the exterior portion 138 is formed.

The interior portion 240 is sized, shaped and configured to be received within the channel 256 of the exterior portion 238 and substantially corresponds in length to the exterior portion 238. Similarly to the interior portion 140, the interior portion 240 is received within the channel 256 of the exterior portion 238 in a substantially curved configuration, which corresponds to an interior surface 248 of the exterior portion 238, and defines longitudinal edges 250 forming a longitudinal slot 258 therebetween. Similarly to the interior portion 140, the interior portion 240 is formed of a material softer than the material of which the exterior portion 238 is formed and, in an exemplary embodiment, is formed of a material configured to be able to form a seal about an element about which it is enclosed. In one example, the interior portion 240 is formed of a plastic material such as, for example, silicone.

In the open configuration, as shown in FIG. 10, the exterior portion 238 and the interior portion 240 are positioned relative to one another so that the longitudinal slot 254 of the exterior portion 238 and the longitudinal slot 258 of the interior portion 240 are aligned with one another so that the connector 110 and the medical device 108 may be passed therethrough to be received within a central portion 260 of the clip 212. In the closed configuration, as shown in FIG. 11A, the exterior portion 238 is rotated relative to the interior portion 240, about a longitudinal axis thereof, to increase a curvature of the interior portion 240 so that longitudinal edges 250 of the interior portion 240 are drawn toward one another. Thus, as the clip 212 is moved toward the closed configuration, the interior portion 240 is pressed about the connector 110 and the medical device 108 to seal the proximal end 120 of the connector 110. The exterior portion 238 may be rotated relative to the interior portion 240 to move the clip 212 between the open and the closed configurations, as desired. In a further exemplary embodiment, as shown in FIG. 11B, the longitudinal edges 250 may be drawn toward one another until the longitudinal edges 250 contact one another and fully enclose the medical device 108 therein.

It will be understood by those of skill in the art that the clip 212 may be utilized in a manner substantially similar to the clip 112 as described above with respect to the system 100. The clip 212, however, should be mounted over a portion of the proximal portion 116 of the connector 110, which extends proximally of the fluid injection port 118 and/or the connecting tube 152, so as not to interfere with the fluid injection port 118 and/or the connecting tube 152 when moving the clip 112 from the open configuration toward the closed configuration.

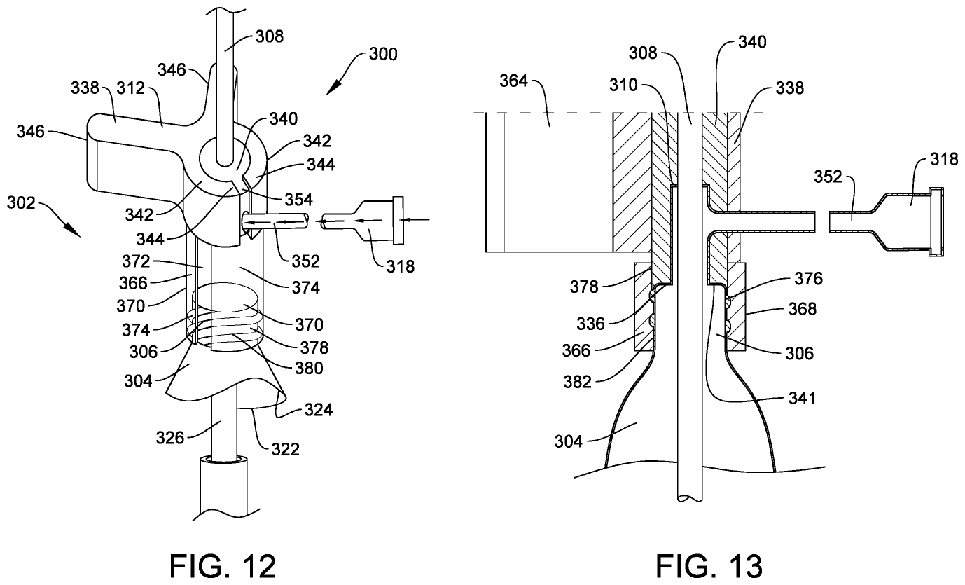

As shown in FIGS. 12-13, a system 300 according to another exemplary embodiment of the present disclosure is substantially similar to the system 100 described above except as detailed below. The system 300 comprises a device 302 (e.g., a luer device) which may be used in a manner substantially similar to the device 102, as described above with respect to the system 100. Similarly to the device 102, the device 302 is configured to be coupled to a proximal end 306 of an access cannula 304 and a medical device 308 received therein. The medical device 308 and the access cannula 304 are substantially similar to the medical device 108 and the access cannula 104, described above with respect to the system 100. The device 302, however, does not require a separate connector 110 component, as described above with respect to the device 102. Rather, as will be described in further detail below, the device 302 includes a fluid path that is built into the device 302 so that any fluid injected through the fluid injection port 318 is directed through the access cannula 304, about the medical device 308, in the space 322 between the interior surface 324 of a channel of the access cannula 304 and the exterior surface 326 of the medical device 308.

The device 302 includes a proximal component 364 and a distal component 366 rotatably coupled to one another so that the distal component 366 is rotatable relative to the proximal component 364 about a longitudinal axis thereof. The proximal component 364 may be configured substantially similarly to the clip 112 described above while the distal component 366 may be configured substantially similarly to the clip 212 described above.

In particular, the proximal component 364, similarly to the clip 112 includes an exterior portion 338 including a pair of jaws 342 curved and connected to one another to define a substantially tubular shape including a longitudinal slot 354 extending along an entire length thereof so that longitudinal edges 344 of the jaws 342 are movable away from one another and toward one another between an open configuration and a closed configuration, respectively. As described above, the shape of the jaws 342 are such that, in a cross-sectional view of the jaws 342, an interior surface 348 extending along the jaws 342 has a substantially circular or C-shape. Similarly to the clip 112, the jaws 342 are biased toward the closed configuration. Also similarly to the clip 112, the exterior portion 338 of the proximal component 364 of the device 302 also includes a pair of wings 346, each of the wings 146 extending laterally from a corresponding one of the jaw 342, in a direction extending away from the longitudinal edges 344 of the jaws 342. Drawing the wings 346 toward one another moves the exterior portion 338 from the closed configuration toward the open configuration, and releasing the wings 346 permits the jaws 342 to revert to their biased closed configuration.

The distal component 366, similarly to the clip 212, includes an exterior portion 368 including a pair of jaws 370 curved and connected to one another to define a substantially tubular shape having a longitudinal slot 372 extending along an entire length thereof so that longitudinal edges 374 of the jaws 370 are separated from one another. In an exemplary embodiment, the jaws 370 are shaped such that, in a cross-sectional view of the jaws 370, an interior surface 376 extending along an interior of the jaws 370 has a substantially circular or C-shape. In this embodiment, a distal portion 378 of the distal component 366 includes a threading 380 extending along the interior surface 376 thereof so that, when the device 302 is clipped over the proximal end 306 of the access cannula 304 and the medical device 308, as will be described in further detail below, the distal component 366 may be rotated relative to the proximal component 364 to be threadedly engaged with the access cannula 304.

According to an exemplary embodiment, the exterior portion 368 of the distal component 366 may be rotatably connected to the exterior portion 338 of the proximal component 364. For example, the exterior portions 368, 338 may have corresponding circumferentially extending tongue and groove connection mechanisms facilitating relative rotation therebetween about a longitudinal axis thereof. Similarly to the clip 212, the distal component 366 may be moved between the open configuration and the closed configuration by rotating the exterior portion 368 relative to an interior portion 340 (and the proximal component 364), as will be described in further detail below.

Similarly to the device 102 and the clip 212 described above, the device 302 includes an interior portion 340 having a correspondingly curved shaped so that the interior portion 340 extends along the interior surfaces 348, 376 of the proximal component 364 and the distal component 366, respectively. The interior portion 340, however, may extend in length along the interior surface 348 of the proximal component 364 and a proximal, non-threaded portion 382 along the interior surface 376 of the distal component 366. Similarly to the interior portions 140, 240 described above, the interior portion 340 is formed of a material softer than the material forming the exterior portions 338, 368 of the proximal and distal components 364, 366, respectively, to form a seal about the medical device 308 and/or access cannula 304.

In an exemplary embodiment, in the open configuration, the interior portion 340 is configured so that a distance between longitudinal edges 350 corresponds to the longitudinal slots 354, 372 of the proximal and distal components 364, 366 so that, desired portions of the medical device 308 and the access cannula 304 may be passed through the longitudinal slots 354, 372 and between the longitudinal edges 350 to be received within the curvature of the interior portion 340, between the jaws 342, 370 of the proximal and distal components 364, 366, respectively. The proximal and distal components 364, 366 may be moved between the open and closed configurations substantially as described above with respect to the clips 112, 212 described above. In particular, the wings 346 of the proximal component 364 may be drawn toward one another so that the proximal component 364 may be placed over the medical device 308 and the distal component 366 positioned over the proximal end 306 of the access cannula 304. Releasing the wings 346 seals the proximal component 364 over the medical device 308.

As will be described in further detail below, upon sealing the proximal component 364 over the medical device 308, the distal component 366 may be rotated relative to the proximal component 364, the interior portion 340, and the access cannula 304, to move the distal component 366 from the open configuration toward the closed configuration, so that the threading 380 of the distal component 366 engages a threading 328 at the proximal end 306 of the access cannula 304. As the exterior portion 368 is rotated relative to the interior portion 340 toward the closed configuration, the longitudinal edges 350 of a portion of the interior portion 340 received therein are drawn toward one another, reducing a cross-sectional area (e.g., diameter) thereof and the distal component 366 is moved distally relative to the access cannula 304 as it is threadedly engaged therewith. Thus, in the closed configuration, the interior portion 340 is pressed and sealed against the proximal end 306 of the access cannula 304.

The device 302 includes the built-in fluid path which, in an exemplary embodiment, is configured as a tubular member 310 housed within the curvature of the interior portion 340, extending between the proximal and distal components 364, 366. The tubular member 310 is in fluid connection with a fluid injection port 318 via, for example, a connecting tube 352 so that, fluid injected via the fluid injection port 318 is directed into and through the tubular member 310.

The tubular member 310 is configured such that, when the proximal component 364 is moved from the open configuration toward the closed configuration in an operative position, the interior portion 340 is pressed and sealed against the medical device 308 and a proximal end 320 of the tubular member 310 so that fluid provided through the tubular member 310 is prevented from escaping proximally therefrom and instead directed distally therethrough and into the space 322 between the interior surface 324 of the access cannula 304 and the exterior surface 326 of the medical device 308. A distal end 336 of the tubular member 310 corresponds to a distal end 341 of the interior portion 340 so that, when the distal component 366 is threadedly engaged with the proximal portion 306 of the access cannula 304, the distal end 341 is pressed and sealed against the proximal end 306 of the access cannula 304 so that fluid through the tubular member 310 is directed into the space 322. The fluid injecting port 318 and the connecting tube 352 are configured so that, when the device 302 is in the closed configuration, the connecting tube 352 and/or the fluid injecting port 318 extend between the longitudinal edges 350 along the interior portion 340.

The device 302 may be utilized in a manner substantially similar to the device 102 of the system 100. While proximal and distal components 364, 366 of the device 302 are in the open configuration, as described above, the device 302 may be placed over the medical device 308 so that the medical device 308 is slidably received within the tubular member 310. The device 302 may be moved distally relative to the medical device 308, in the open configuration, until the distal component 366 is positioned over the proximal end 306 of the access cannula 304. In this position, the proximal component 364 may be moved toward the closed configuration (e.g., by releasing the wings 346 and allowing the exterior portion 338 to revert to its biased configuration). In the closed configuration, the proximal component 364 is sealed over the medical device 308 and the proximal end 320 of the tubular member 310.

Once the proximal component 364 is in the closed configuration, the distal component 366 may be moved toward the closed configuration by rotating the distal component 366 relative to the proximal component 364 (and relative to the interior portion 340 and the access cannula 304) so that threads 380 of the distal component 366 engage the threading 328 of the proximal end 306 of the access cannula 304. As described above, closing of the distal component 366 seals the interior portion 340 about the tubular member 310 and presses the tubular member 310 against the proximal end 306 of the access cannula 304 so that fluid received therein is directed through the access cannula 304, as described above.

According to another exemplary embodiment, rather than having the exterior portions 338, 368 of the proximal and distal components 364, 366, respectively, rotatably connected one another, the exterior portions 338, 368 of the proximal and distal components 364, 366 may be separable from one another and from the interior portion 340. Thus, upon placement of the interior portion 340 and the tubular member 310 over the medical device 308, as desired, the proximal and distal components 364, 366 may be independently clipped thereover.

In particular, the interior portion 340 may be placed over a portion of the medical device 308 such that the distal end 341 of the interior portion is in contact with and/or distally pressed against the proximal end 306 of the access cannula 304. The proximal component 364 may then be clipped over a proximal portion of the interior portion 340, including the proximal end of the tubular member 310 housed therein, and the distal component 366 may be clipped over the distal end 341 of the interior portion 340 and a portion of the proximal end 306. In an exemplary embodiment, the distal component 366 may be mounted over the interior portion 340 and the proximal end 306 and rotated relative thereto, about a longitudinal axis thereof, so that as the threads 380 of the distal component 366 threadedly engage the threading 328 along the proximal end 306, the interior portion 340 is sealed against the distal end 336 of the tubular member 310 and the proximal end 306 of the access cannula 304.

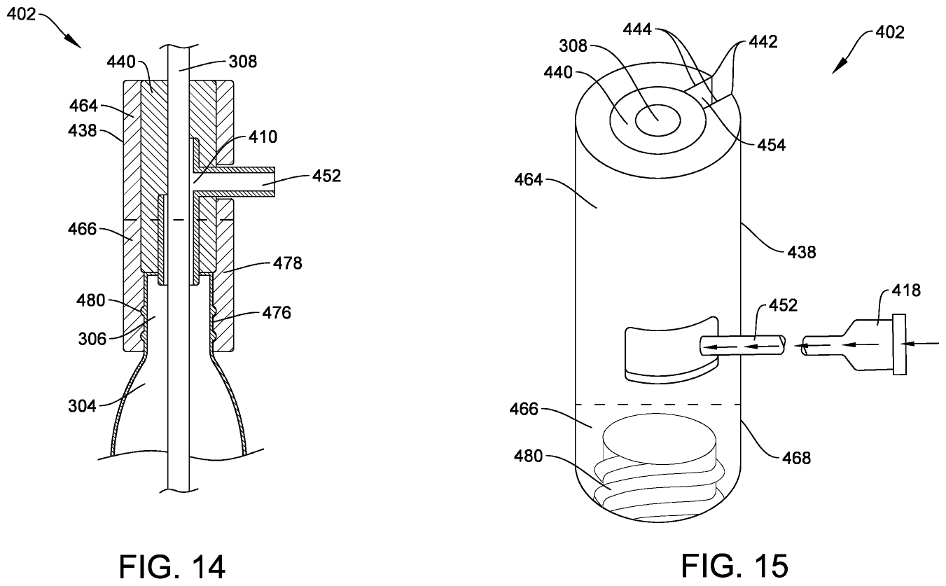

As shown in FIGS. 14-15, a device 402 (e.g., a luer device) according to another exemplary embodiment is substantially similar to the device 302, described above, and may be used in place of the device 302 according to the system 300. Similarly to the device 302, the device 402 is configured to be coupled to the proximal end 306 of the access cannula 304 and the medical device 308 received therein. The device 402 includes an interior portion 440 extending from a first end to a second end, a tubular member 410 housed within a curvature of the interior portion 440, a proximal component 464 and a distal component 466. The interior portion 440 and the tubular member 410 are configured to receive the medical device 308 in a substantially similar manner as the interior portion 340 and the tubular member 310 described above.

Similarly to the tubular member 310, the tubular member 410 is in fluid connection with a fluid injection portion 418 via, for example, a connecting tube 452 so that, fluid injected via the fluid injection port 418 is directed into and through the tubular member 410. Further, the proximal component 464 and the distal component 466 are configured to be rotatably coupled to one another so that the distal component 466 is rotatable relative to the proximal component 464 about a longitudinal axis thereof. Each of the proximal component 464 and the distal component 466 may be configured substantially similarly to the clip 212 described above.

In particular, the proximal component 464, similarly to the clip 212 includes an exterior portion 438 including a pair of jaws 442 curved and connected to one another to define a substantially tubular shape including a longitudinal slot 454 extending along an entire length thereof so that longitudinal edges 444 of the jaws 442 are movable away from one another and toward one another between an open configuration and a closed configuration, respectively. Further, the distal component 466, similarly to the distal component 366, includes an exterior portion 468 including a pair of jaws (not shown) curved and connected to one another to define a substantially tubular shape having a longitudinal slot (not shown) extending along an entire length thereof so that longitudinal edges (not shown) of the jaws (not shown) are separated from one another.

Moreover, a distal portion 478 of the distal component 466 includes a threading 480 extending along an interior surface 476 thereof so that, when the device 402 is clipped over the proximal end 306 of the access cannula 304 and the medical device 308. The distal component 466 may be rotated relative to the proximal component 464 to be threadedly engaged with the access cannula 304, in a substantially similar manner as the distal component 366 and the proximal component 364 described above. Although the distal component 466 and the proximal component 464 may be configured to rotate independently relative to each other, in a further embodiment, the distal component 466 and the proximal component 464 may be configured to rotate together simultaneously. For example, in the further embodiment, the distal component 466 and the proximal component 464 may be coupled to one another via a coupling mechanism such that the distal component 466 and the proximal component 464 may be rotated together in a singular motion. In a yet further embodiment, the distal component 466 and the proximal component 464 may be locked to one another via a locking mechanism and rotated together in a singular motion.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

1-15. (canceled)

16. A luer device for accessing a biliary duct, comprising:
a connector including a body extending longitudinally from a proximal end to a distal end and including a lumen extending therethrough, the body of the connector including a distal portion sized, shaped, and configured to be coupled to a proximal end of an access cannula and a proximal portion of the body sized, shaped, and configured to slidably receive a medical device received within the access cannula; and
a clip configured to be clipped over the proximal portion of the connector and a portion of the medical device extending therefrom, the clip including an exterior portion and an interior portion, the exterior portion extending along a longitudinal axis from a proximal end to a distal end and including a channel extending therethrough along the longitudinal axis, a longitudinal slot extending along an entire length of the exterior portion so that the channel is open to and in communication with an exterior of the clip via the longitudinal slot, the interior portion extending along an interior surface of the channel defining jaws and formed of a material which, when the clip is moved from an open configuration toward a closed configuration, in an operative position, is pressed against the medical device and the connector to seal the proximal end of the connector and direct fluid distally therethrough and through a space extending between an interior surface of the access cannula and an exterior surface of the medical device.

17. The device of claim 16, wherein the longitudinal slot defines a pair of jaws movable between the open configuration, in which longitudinal edges defining the jaws are moved away from one another, and the closed configuration, in which the longitudinal edges are moved toward one another to draw longitudinal edges of the interior portion toward one another to seal the proximal end of the connector.

18. The device of claim 17, wherein the clip is biased toward the closed configuration.

19. The device of claim 18, wherein the exterior portion of the clip includes a pair of wings, each of the wings extending laterally from a corresponding one of the jaws, in a direction away from the longitudinal edges of the jaws so that, when the pair of wings are drawn toward one another the jaws are moved away from one another toward the open configuration, and when the pair of wings are released, the jaws are permitted to revert toward the closed configuration.

20. The device of claim 16, wherein the interior portion is formed of a softer material then the exterior portion of the clip.

21. The device of claim 16, wherein the connector includes a fluid connection port in fluid connection with the lumen of the connector via a connecting tube so that fluid injected via the fluid connection port.

22. The device of claim 16, wherein the proximal portion of the connector has a smaller cross-sectional area than the distal portion of the connector.

23. The device of claim 16, wherein the distal portion of the connector includes a threading along an interior surface thereof for threadedly engaging a corresponding threading along the proximal end of the access cannula.

24. The device of claim 16, wherein the exterior portion of the clip is rotatable relative to the interior portion to move the clip from the open configuration toward the closed configuration.

25. A luer device, comprising:
a proximal component extending along a longitudinal axis from a proximal end to a distal end and including a channel extending therethrough, a longitudinal slot extending along an entire length of the proximal component so that the channel thereof is open to and in communication with an exterior of the proximal component via the longitudinal slot, the longitudinal slot defining a pair of jaws movable between an open configuration, in which longitudinal edges defining the jaws are moved away from one another, and a closed configuration, in which the longitudinal edges are moved toward one another;
a distal component rotatably longitudinally aligned with and rotatably coupled to the proximal component so that the distal component is rotatable relative thereto about the longitudinal axis thereof, the distal component extending from a proximal end to a distal end and including a channel extending therethrough, a longitudinal slot extending along an entire length of the distal component so that the channel thereof is open to and in communication with an exterior of the distal component via the longitudinal slot thereof, a distal portion of the distal component including a threading along an interior thereof for threadedly engaging the proximal end of an access cannula;
an interior portion extending along an interior surface of the channel of the proximal component defining the jaws and along an interior surface along an unthreaded proximal portion of the distal component, the interior portion formed of a material which, when pressed against a medical device, forms a seal thereabout; and
a tubular member received within the interior portion, along a distal portion thereof, a distal end of the tubular member corresponding to a distal end of the interior portion so that, when the proximal portion is in the closed configuration in an operative configuration over the medical device, and the distal component is coupled to the access cannula and the medical device is received within the tubular member, a proximal end of the tubular member is sealed and fluid is directed distally through an access channel, about the medical device.

26. The device of claim 25, wherein, the interior portion extends along the interior surface of the proximal component so that, when the proximal portion is in the open configuration longitudinal edges of the interior portion are separated from one another and, when the proximal portion is in the closed configuration, longitudinal edges of the interior portion are drawn toward one another.

27. The device of claim 25, wherein the proximal component includes a pair of wings, each of the wings extending laterally from a corresponding one of the jaws, in a direction away from the longitudinal edges of the jaws so that, when the pair of wings are drawn toward one another the jaws are moved away from one another toward the open configuration, and when the pair of wings are released, the jaws are permitted to revert toward the closed configuration.

28. The device of claim 25, wherein the interior portion is formed of a softer material than an exterior portion of a clip.

29. The device of claim 25, further comprising a fluid connection port in fluid connection with the tubular member, the fluid connection port configured to extend exterior to the proximal component, when the proximal component is in the closed configuration.

30. The device of claim 25, wherein the distal component is rotatable relative to the interior portion so that, as the distal component is moved distally relative to the access cannula during a threaded engagement therebetween, the distal end of the interior portion is pressed distally against the proximal end of the access cannula to direct fluid flow distally through the tubular member and distally through a space extending between an interior of the access channel and an exterior surface of the medical device.

31. A method for treating a biliary duct, comprising:
coupling a connector to a proximal end of an access cannula, which includes a medical device housed therein and a distal end which has been inserted into a target duct, such that a distal portion of the connector is engaged with the proximal end of the access cannula and a portion of the medical device extending proximally from the access cannula is slidably received within a proximal portion of the connector;
clipping a clip over a proximal portion of the connector and a portion of the device extending proximally therefrom, the clip including an interior portion which, when the clip is in a clipped configuration, is pressed against the medical device and the connector to seal the proximal end of the connector; and
injecting a fluid, via a fluid injection port that is in fluid communication with the connector, into a channel of the connector, the fluid being directed distally through the connector, since the proximal end of the connector is sealed, and distally through a space extending between an interior surface of an access channel and an exterior surface of the medical device.

32. The method of claim 31, further comprising confirming, via fluoroscopic visualization, whether the distal end of the access cannula is positioned within the target duct, as desired.

33. The method of claim 31, wherein clipping the clip includes moving the clip from an open configuration toward a closed configuration, the clip including an exterior portion extending along a longitudinal axis from a proximal end to a distal end and including a channel extending therethrough along the longitudinal axis, a longitudinal slot extending along an entire length of the exterior portion so that the channel is open to and in communication with an exterior of the clip via the longitudinal slot, the interior portion extending along an interior surface of the channel defining jaws and formed of a material which, when the clip is moved from an open configuration toward a closed, clipping configuration, longitudinal edges of the interior portion are drawn toward one another and the interior portion is pressed against the medical device and the connector received therein to form a seal thereabout.

34. The method of claim 33, wherein moving the clip between the open and closed configurations includes drawing a pair of wings, each of which extend laterally from a corresponding one of the jaws, toward one another to move the jaws of the clip away from one another toward the open configuration, and releasing the wings so that the jaws are permitted to revert toward the closed configuration.

35. The method of claim 31, wherein the medical device is one of a needle and a guidewire.
